(19) United States
(12) Patent Application Publication
(10) Pub. No.: US 2005/0226943 A1
(43) Pub. Date: Oct. 13, 2005

(54) Extract of sophora flavescens flavonoids and uses thereof

(76) Inventors: Xiaoqiang Yan, Shanghai (CN); Yumin Cui, Shanghai (CN); Tao Wang, Shanghai (CN); Zhiming Ma, Shanghai (CN); Ke Pan, Shanghai (CN); Weihan Zhang, Shanghai (CN); Weihui Huang, Shanghai (CN); Jianrong Hong, Shanghai (CN); Jeff Duan, Shanghai (CN); Yu Cai, Shanghai (CN)

Correspondence Address: EDWARDS & ANGELL, LLP, P.O. BOX 55874, BOSTON, MA, 02205, US

(21) Appl. No.: 11/095,783

(22) Filed: Mar. 30, 2005

Related U.S. Application Data
(60) Provisional application No. 60/558,901, filed on Apr. 1, 2004.

(51) Int. Cl. A61K 35/78; A61K 31/7048; A61K 31/353

(52) U.S. Cl. 424/725; 514/27; 514/456

(57) ABSTRACT

Compositions containing a Sophora flavescens flavonoids extract or its active ingredients, Kurarinone, 2′-methoxy-Kurarinone, and Sophoraflavanone G are described. Also disclosed is a method of treating TNFα and IL-1β related diseases using such compositions.

### CROSS-REFERENCE TO RELATED APPLICATIONS

This application is a utility application of and claims benefit of U.S. Provisional Application Ser. No. 60/558,901, filed Apr. 1, 2004, which is incorporated by reference in its entirety.

### FIELD OF INVENTION

This invention is in the field of medicinal chemistry. Particularly, it relates to the extract of Sophora flavescens flavonoids and the use of this composition as a therapeutically effective agent for treating, preventing, or ameliorating TNFα and IL-1β related diseases.

### BACKGROUND

Inflammation is a defensive reaction in response to injury induced by a variety of inflammatory factors in the human body. The local signs of inflammation typically include hyperemia, edema, heat, and pain. These signs are more obvious in acute inflammation than those in chronic inflammation or splanchnic inflammation. In addition to local response, inflammation can induce systemic response such as fever, leukocytosis, and degeneration or necrosis and the like may occur in some solid organs such as the heart, liver or lungs to various extents.

According to pathologic classification, inflammation falls into several categories including: alterative inflammation, serious inflammation, fibrinous inflammation, purulent inflammation, hemorrhagic inflammation, catarrhal inflammation, proliferative inflammation, and chronic granulomatous inflammation.

TNFα, a mononuclear cytokine, is produced by monocytes and macrophages chiefly. Lipopolysaccharide (“LPS”) is a potent stimulator for it. TNFα was identified as possessing various bioactivities including: a) killing or inhibition of cancer cells; b) enhancement of the phagocytosis of neutrophilic granulocyte, up-regulation of the production of peroxide negion, and involvement in inflammatory reaction; c) anti-infection; etc.

It was disclosed that TNFα production inhibitors had been used to treat a variety of disorders, such as rheumatoid arthritis, juvenile rheumatoid arthritis, osteoarthritis, spondyloarthropathies, inflammatory bowel disease, chronic heart failure, diabetes mellitus, systemic lupus erythematosus, scleroderma, sarcoidosis, polymyositis/dermatomyositis, psoriasis, multiple myeloma, myelodysplastic syndrome, acute myelogenous leukemia, Parkinson's disease, AIDS dementia complex, Alzheimer's disease, depression, sepsis, pyoderma gangrenosum, hematosepsis, septic shock, Behcet's syndrome, graft-versus-host disease, uveitis, Wegener's granulomatosis, Sjogren's syndrome, chronic obstructive pulmonary disease, asthma, acute pancreatitis, periodontal disease, cachexia, cancer, central nervous system injury, viral respiratory disease, obesity, etc.⁽¹⁻²⁵⁾ (1. Ogata H, Hibi T. et al Curr Pharm Des. 2003; 9(14): 1107-13. 2. Moller D R. et al J Intern Med. 2003 January; 253(1): 31-40. 3. Taylor P C. Et al Curr Pharm Des. 2003; 9(14): 1095-106. 4. Wilkinson N et al Arch Dis Child. 2003 March; 88(3): 186-91. 5. Nishimura F et al J Periodontol. 2003 January; 74(1): 97-102. 6. Weinberg J M et al Cutis. 2003 January; 71(1): 41-5. 7. Burnham E et al Crit Care Med. 2001 March; 29(3): 690-1. 8. Sack M. et al Pharmacol Ther. 2002 April-May; 94(1-2): 123-35. 9. Barnes P J. et al Annu Rev Pharmacol Toxicol. 2002; 42:81-98. 10. Mageed R A et al Lupus. 2002; 11(12): 850-5. 11. Tsimberidou A M et al Expert Rev Anticancer Ther. 2002 June; 2(3): 277-86. 12. Muller T. et al Curr Opin Investig Drugs. 2002 December; 3(12): 1763-7. 13. Calandra T et al Curr Clin Top Infect Dis. 2002; 22:1-23. 14. Girolomoni G et al Curr Opin Investig Drugs. 2002 November; 3(11): 1590-5. 15. Tutuncu Z et al Clin Exp Rheumatol. 2002 November-December; 20(6 Suppl 28): S146-51. 16. Braun J et al Best Pract Res Clin Rheumatol. 2002 September; 16(4): 631-51. 17. Barnes P J. Et al Novartis Found Symp. 2001; 234:255-67; discussion 267-72. 18. Brady M, et al Baillieres Best Pract Res Clin Gastroenterol. 1999 July; 13(2): 265-89. 19. Goldring M B. et al Expert Opin Biol Ther. 2001 September; 1(5): 817-29. 20. Mariette X. Rev Prat. 2003 March 1; 53(5): 507-11. 21. Sharma R et al Int J Cardiol. 2002 September; 85(1): 161-71. 22. Wang C X et al Prog Neurobiol. 2002 June; 67(2): 161-72. 23. Van Reeth K et al Vet Immunol Immunopathol. 2002 September 10; 87(3-4): 161-8. 24. Leonard B E et al Int J Dev Neurosci. 2001 June; 19(3): 305-12. 25. Hays S J et al Curr Pharm Des. 1998 August; 4(4): 335-48.).

IL-1β is a cytokine produced by monocyte macrophages, dendritic cells, fibroblasts and so on, which can stimulate the proliferation and differentiation of T-cells or B-cells, stimulate hematopoiesis, and is involved in inflammatory reaction. It was disclosed that IL-1β production inhibitors had been used to treat a variety of diseases such as rheumatoid arthritis, hematosepsis, periodontal disease, chronic heart failure, polymyositis/dermatomyositis, acute pancreatitis, chronic obstructive pulmonary disease, Alzheimer's disease, osteoarthritis, bacterial infections, multiple myeloma, myelodysplastic syndrome, uveitis, central nervous system injury, viral respiratory disease, asthma, depression, scleroderma etc^{(26˜45)} (26. Taylor P C. et al Curr Pharm Des. 2003; 9(14): 1095-106. 27. Dellinger R P et al Clin Infect Dis. 2003 May 15; 36(10): 1259-65. 28. Takashiba S et al J Periodontol. 2003 January; 74(1): 103-10. 29. Diwan A, et al Curr Mol Med. 2003 March; 3(2): 161-82. 30. Lundberg I E, et al Rheum Dis Clin North Am. 2002 November; 28(4): 799-822. 31. Makhija R, et al J Hepatobiliary Pancreat Surg 2002; 9(4): 401-10. 32. Chung K F. Et al Eur Respir J Suppl. 2001 December; 34:50s-59s. 33. Hallegua D S, et al Ann Rheum Dis. 2002 November; 61(11): 960-7. 34. Goldring M B. Et al Expert Opin Biol Ther. 2001 September; 1(5): 817-29. 35. Mrak R E, Griffin W S. Et al Neurobiol Aging. 2001 November-December; 22(6): 903-8. 36. Brady M, et al Baillieres Best Pract Res Clin Gastroenterol. 1999 July; 13(2): 265-89. 37. Van der Meer J W, et al Ann NY Acad Sci. 1998 September 29; 856:243-51. 38. Rameshwar P et al Acta Haematol. 2003; 109(1): 1-10. 39. de Kozak Y et al Int Rev Immunol. 2002 March-June; 21(2-3): 231-53. 40. Wang C X et al Prog Neurobiol. 2002 June; 67(2): 161-72. 41. Van Reeth K et al Vet Immunol Immunopathol. 2002 September 10; 87(3-4): 161-8. 42. Stirling R G et al Br Med Bull. 2000; 56(4): 1037-53. 43. Leonard B E et al Int J Dev Neurosci. 2001 June; 19(3): 305-12. 44. Allan S M et al Ann NY Acad Sci. 2000; 917:84-93. 45. Cafagna D et al Minerva Med. 1998 May; 89(5): 153-61.)

Sophora flavescens Ait is known in the art of Chinese folk medicines. Two types of compounds have been found and isolated from this herb as the main biologically active components. They are Sophora flavescens alkaloids and Sophora flavescens flavonoids, respectively. To date, 23 alkaloids, and 32 flavonoids or isoflavonoids have been identified from the root, leaves or flowers of Sophora flavescens A (Miao Likang, Zhang Jianzhong, et al., Natural Product Research and Development, 2000, 13(2): 69-73).

Kurarinone, 4H-1-Benzopyran-4-one, 2-(2,4-dihydroxyphenyl)-2,3-dihydro-7-hydroxy-5-methoxy-8-[5-methyl-2-(1-methylethenyl)-4-hexenyl]-, can be obtained from Sophora flavescens Ait and Gentiana macrophylla Pall chiefly. Its structure is as follows:

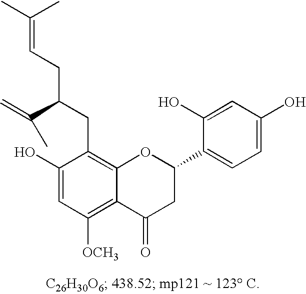

2′-methoxy-Kurarinone (Isokurarinone), is a yellow crystal, the structure of which is as follows:

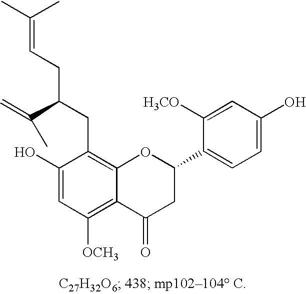

Sophoraflavanone G (Norkurarinone), is a colorless needle (benzene), the structure of which is as follows:

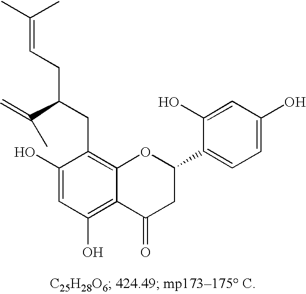

The inhibitory activity against TNFα or of Kurarinone, 2′-methoxy-Kurarinone or Sophoraflavanone G has not been disclosed yet.

### SUMMARY

This invention relates to an extract of Sophora flavescens flavonoids, its isolated active ingredients (Kurarinone, 2′-methoxy-Kurarinone or Sophoraflavanone G), and its use for treating, preventing, or ameliorating TNFα and IL-1β related diseases or disease symptoms. In one aspect, the invention features an extract of Sophora flavescens flavonoids. The extract includes (or consists essentially of) 20˜60% by weight Kurarinone, 1˜5% by weight 2′-methoxy-Kurarinone, and 1˜12% by weight Sophoraflavanone G. In particular, the extract may include (or consist essentially of) 45% by weight Kurarinone, 2% by weight 2′-methoxy-Kurarinone, and 6% by weight Sophoraflavanone G.

The method of producing the extract (e.g., as described herein) is also within the invention.

In another aspect, the invention provides a composition consisting essentially of at least two members of Kurarinone, 2′-methoxy-Kurarinone, or Sophoraflavanone G. Also within the invention is a composition containing (or consisting essentially of) at least two members of Kurarinone, 2′-methoxy-Kurarinone, or Sophoraflavanone G, wherein at least a portion of one of the members in the composition is isolated (e.g., one, two or three members are isolated). Such compositions may be produced, for example, by combining at least two members of non-isolated Kurarinone, 2′-methoxy-Kurarinone, or Sophoraflavanone G; or isolated Kurarinone, 2′-methoxy-Kurarinone, or Sophoraflavanone G.

A pharmaceutically acceptable carrier can be added to a Sophora flavescens flavonoids extract of the invention or its active ingredients to produce a pharmaceutical composition. Examples of such compositions include: a composition containing isolated Kurarinone and a pharmaceutically acceptable carrier; a composition containing isolated 2′-methoxy-Kurarinone and a pharmaceutically acceptable carrier; a composition containing isolated Sophoraflavanone G and a pharmaceutically acceptable carrier; a composition consisting essentially of a pharmaceutically acceptable carrier and at least two members of Kurarinone, 2′-methoxy-Kurarinone, or Sophoraflavanone G; and a composition containing (or consisting essentially of) a pharmaceutically acceptable carrier and at least two members of Kurarinone, 2′-methoxy-Kurarinone, or Sophoraflavanone G, wherein at least a portion of one of the members in the composition is isolated (e.g., one, two or three members are isolated).

The compositions can further include additional active agents. The composition can additionally include other therapeutic agents, including anticancer agents (antiproliferation agents, chemotherapeutics), antianemia agents, antinausea agents, antiangiogenesis agents, or anti-inflammatory agents, agents that modulate TNFα and/or IL-1β.

The third aspect of the present invention is to provide a method for treating, preventing or ameliorating TNFα or IL-1β related disorders by administering an effective amount of the Sophora flavescens flavonoids extract, its active ingredients (alone or in combination), and the compositions delineated herein to a subject in need of such treatment. The subject (e.g., a mammal such as human) may be one who is identified as being in need of inhibition of TNFα or IL-1β production. In another aspect, the methods described herein can further include the step of identifying the subject as in need of a specific treatment (e.g., a disorder, disease or disease symptom delineated herein). Identifying a subject in need of such treatment can be in the judgment of a subject or a health care professional and can be subjective (e.g., opinion) or objective (e.g., measurable by a test or diagnostic method).

Examples of the TNFα related disorders to be treated, prevented or ameliorated include, but are not limited to, rheumatoid arthritis, juvenile rheumatoid arthritis, osteoarthritis, spondyloarthropathies, inflammatory bowel disease, chronic heart failure, diabetes mellitus, systemic lupus erythematosus, scleroderma, sarcoidosis, polymyositis/dermatomyositis, psoriasis, multiple myeloma, myelodysplastic syndrome, acute myelogenous leukemia, Parkinson's disease, AIDS dementia complex, Alzheimer's disease, depression, sepsis, pyoderma gangrenosum, hematosepsis, septic shock, Behcet's syndrome, graft-versus-host disease, uveitis, Wegener's granulomatosis, Sjogren's syndrome, chronic obstructive pulmonary disease, asthma, acute pancreatitis, periodontal disease, cachexia, cancer, central nervous system injury, viral respiratory disease, and obesity.

Examples of the IL-1β related disorders to be treated, prevented or ameliorated include, but are not limited to, rheumatoid arthritis, hematosepsis, periodontal disease, chronic heart failure, polymyositis/dermatomyositis, acute pancreatitis, chronic obstructive pulmonary disease, Alzheimer's disease, osteoarthritis, bacterial infections, multiple myeloma, myelodysplastic syndrome, uveitis, central nervous system injury, viral respiratory disease, asthma, depression, and scleroderma.

In another aspect, the method further includes administering a compound or extract delineated herein to a subject according to instructions listed on a label or package insert accompanying a container having therein the compound or composition thereof according to any of the formulae herein. The instructions can include administration information, including dosage, route of administration, side effects or contraindication situations. The instructions can include information required by a regulatory agency (e.g., Food and Drug Administration or other similar regulatory agency).

The details of one or more embodiments of the invention are set forth in the accompanying drawings and description below. Other features, objects, and advantages of the invention will be apparent from the drawings and the description, and from the claims.

### BRIEF DESCRIPTION OF THE DRAWINGS

FIG. 1. Inhibition of Kurarinone (Kus a) and Sophora flavescens flavonoids extract (0912) on LPS-induced expression of TNFα.

FIG. 2. Inhibition of Kurarinone (Kus a) and Sophora flavescens flavonoids extract (0912) on LPS-induced expression of IL1-β.

FIG. 3. HPLC of Sophora flavescens flavonoids.

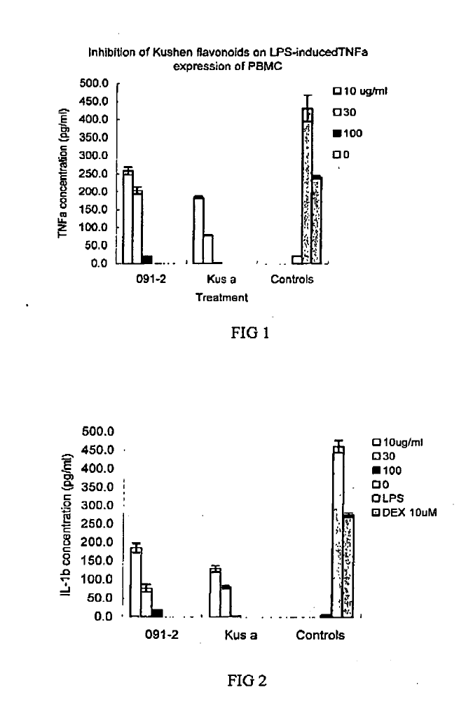

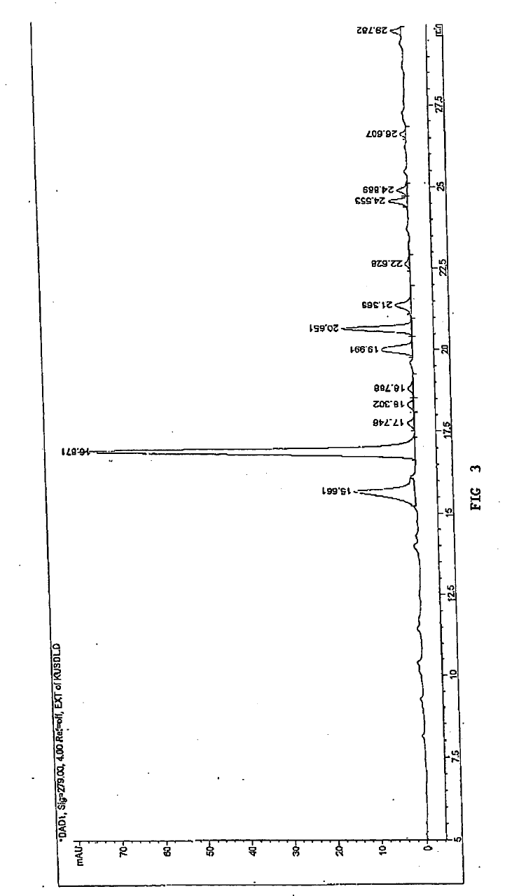

### DETAILED DESCRIPTION

This invention is based in part on an unexpected discovery that Kurarinone, 2′-methoxy-Kurarinone, and Sophoraflavanone G are present in a Sophora flavescens flavonoids extract which has TNF-α and IL1-β production inhibitory activities. Thus, the Sophora flavescens flavonoids extract and its active ingredients (alone or in combination) are useful for treating TNF α or IL-1β related disorders.

A Sophora flavescens flavonoids extract of the invention contains 20˜60% (e.g., 45%) by weight Kurarinone, 1˜5% (e.g., 2%) by weight 2′-methoxy-Kurarinone, and 1˜12% (e.g., 6%) by weight Sophoraflavanone G. Preferably, the extract contains 35˜45% by weight Kurarinone, 2˜4% by weight 2′-methoxy-Kurarinone, and 5˜8% by weight Sophoraflavanone G.

The extract can be prepared according to the method of the invention described in Example 1 below. e.g., the plant material of Sophora flavescens Ait is extracted using organic solvent such as ethanol, ethyl acetate etc., then the extracts evaporated in vacuo to dryness, dissolved in 5%˜50% C₁˜C₄ alcohol aqueous solution, then extracted with organic solvent and concentrated, if desired. The said C₁˜C₄ alcohol aqueous solution is methanol, ethanol, propanol, iso-propanol or butanol. Preferably, the concentration of said C₁˜C₄ alcohol aqueous solution is 20˜40%. Most preferably, the concentration is about 30%.

As the biological functions of Kurarinone, 2′-methoxy-Kurarinone, and Sophoraflavanone G differ, the proportion of each ingredient in the Sophora flavescens flavonoids extract may be adjusted as needed, e.g., by further purification of the extract, or by adding to the extract an isolated (including chemically synthesized) ingredient. Alternatively, a composition may be formed by mixing two or three of the isolated ingredients in desired ratios.

As used herein, the term “isolated” refers to a state in which a compound has a higher purity or homogeneity than in its natural state (i.e., root) when obtained from an extract or is subjected to at least one purification process (e.g., crystallization, chromatography, distillation, sublimation, etc.) in order to provide a purer form of the compound. More specifically, isolated Kurarinone is at least 41% (i.e., any integer % between 41% and 100%, inclusive) pure by dry weight, isolated 2′-methoxy-Kurarinone is at least 6% (i.e., any integer% between 6% and 100%, inclusive) pure by dry weight, and isolated Sophoraflavanone G is at least 11% (i.e., any integer % between 11% and 100%, inclusive) pure by dry weight. Purity of a compound can be measured by any appropriate standard method, e.g., by column chromatography or HPLC analysis.

Typically, a pharmaceutically acceptable carrier is added to the Sophora flavescens flavonoids extract or its active ingredients to facilitate their administration. Preferably, the composition contains 0.1-99.9% by weight the extract or its active ingredients. A “pharmaceutically acceptable carrier” does not substantially adversely affect the pharmacological activities of the extract or its ingredients, and is non-toxic when administered in doses sufficient to deliver an effective amount of the extract or its ingredients.

Pharmaceutically acceptable carriers that may be used include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of the extract or its active ingredients.

Other pharmaceutically acceptable additives such as fillers (e.g., anhydrous lactose, starch, lactose beads, and glucose), binders (e.g., microstalline cellulose), disintegrating agents (e.g., cross-linked sodium carboxymethyl starch, low-substituted hydroxypropyl cellulose, and cross-linked PVP), lubricating agents (e.g., magnesium stearate), absorption-promoting agents, flavoring agents, sweetening agents, diluting agents, excipients, wetting agents, solvents, solublizing agents, and coloring agents may also be included in the composition.

The Sophora flavescens flavonoids extract, its active ingredients, and the compositions delineated herein may be administered parenterally or non-parenterally. For oral administration, the Sophora flavescens flavonoids extract, its active ingredients, and the compositions may be in the form of pills, granules, capsules, suspensions, or solutions. For parenteral administration, the Sophora flavescens flavonoids extract, its active ingredients, and the compositions may be in the form of injectable suspensions, creams, ointments, patches, or sprays. The term “parenteral,” as used herein, includes subcutaneous, intracutaneous, intravenous, intramuscular, intraarticular, intraarterial, intrasynovial, intrasternal, intrathecal, intralesional, and intracranial injection or infusion techniques. Other administration routes include oral, topical, rectal, nasal, buccal, vaginal, sublingual, intradermal, mucosal, intratracheal, or intraurethral routes. The Sophora flavescens flavonoids extract, its active ingredients, and the compositions may also be administered via inhalation spray or an implanted reservoir, or through an acupuncture point.

For oral administration, the Sophora flavescens flavonoids extract, its active ingredients, and the compositions may be in any orally acceptable dosage form including, but not limited to, capsules, tablets, emulsions and aqueous suspensions, dispersions, solutions, microcapsules, pills, lozenges, granules, and powders. In the case of tablets, carriers which are commonly used include lactose and corn starch. Lubricating agents, such as magnesium stearate, are also typically added. For oral administration in a capsule form, useful diluents include lactose and dried cornstarch. When aqueous suspensions and/or emulsions are administered orally, the Sophora flavescens flavonoids extract or its active ingredients may be suspended or dissolved in an oily phase and combined with emulsifying and/or suspending agents. If desired, certain sweetening and/or flavoring and/or coloring agents may be added.

The Sophora flavescens flavonoids extract, its active ingredients, and the compositions of the invention may be in the form of a sterile injectable preparation, for example, as a sterile injectable aqueous or oleaginous suspension. This suspension may be formulated according to techniques known in the art using suitable dispersing or wetting agents (such as Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The Sophora flavescens flavonoids extract, its active ingredients, and the compositions of the invention may also be administered in the form of suppositories for rectal administration. These compositions can be prepared by mixing the Sophora flavescens flavonoids extract or its active ingredients with a suitable non-irritating excipient which is solid at room temperature but liquid at the rectal temperature and therefore will melt in the rectum to release the active components. Such materials include, but are not limited to, cocoa butter, beeswax, and polyethylene glycols.

Topical administration of the Sophora flavescens flavonoids extract, its active ingredients, and the compositions of the invention are useful when the desired treatment involves areas or organs readily accessible by topical application. For application topically to the skin, the Sophora flavescens flavonoids extract or its active ingredients should be formulated with a suitable ointment containing the active components suspended or dissolved in a carrier. Carriers for topical administration of the Sophora flavescens flavonoids extract or its ingredients include, but are not limited to, mineral oil, liquid petroleum, white petroleum, propylene glycol, polyoxyethylene or polyoxypropylene compound, emulsifying wax, and water. Alternatively, the composition can be formulated with a suitable lotion or cream containing the active components suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol, and water. The extract or its active ingredients may also be topically applied to the lower intestinal tract by rectal suppository formulation or in a suitable enema formulation. Topically transdermal patches are also included in this invention.

The Sophora flavescens flavonoids extract, its ingredients, and the compositions of the invention may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The Sophora flavescens flavonoids extract, its active ingredients, and the compositions of the invention can be administered using an implantable device. Implantable devices and related technology are known in the art and are useful as delivery systems where a continuous, or timed-release delivery of the extract, its ingredients, or the compositions is desired. Additionally, the implantable device delivery system is useful for targeting specific points of delivery (e.g., localized sites and organs). See, e.g., Negrin et al., Biomaterials 22(6):563, 2001. Timed-release technology involving alternate delivery methods can also be used in this invention. For example, timed-release formulations based on polymer technologies, sustained-release techniques and encapsulation techniques (e.g., polymeric and liposomal) can also be used for delivery of the extract, its ingredients, or the compositions delineated herein.

Also within the invention is a patch to deliver the Sophora flavescens flavonoids extract, its ingredients, or the compositions delineated herein. A patch includes a material layer (e.g., polymeric, cloth, gauze, and bandage) and the compositions of the invention. One side of the material layer can have a protective layer adhered to it to resist passage of the extract, its ingredients, or the compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure-sensitive, i.e., it can allow for positioning of the adhesive (and the device to be adhered to the skin) against the skin by the application of pressure (e.g., pushing and rubbing) on the adhesive or device.

When the Sophora flavescens flavonoids extract, its active ingredients, the compositions, and the formulations of the invention are combined with one or more additional therapeutic or prophylactic agents, both the additional agent and the Sophora flavescens flavonoids extract, its ingredients, and the composition should be present at dosage levels of between about 1 to 100%, and more preferably between about 5 to 95% of the dosage normally administered in a monotherapy regimen. The additional agents may be administered separately, as part of a multiple dose regimen, from the extract, its ingredients, or the compositions of this invention. Alternatively, those agents may be part of a single dosage form, mixed together with the extract, its ingredients, or the composition of this invention in a single formulation.

Packaged products can be manufactured by disposing the Sophora flavescens flavonoids extract, its active ingredients, the compositions and the formulations delineated herein in a container (e.g., bottle, canister, tube, tin, etc.), and printing dosing information on a legend (e.g., a label or product insert) associated with the container. The container is made of any material suitable for holding the extract, its active ingredients, the compositions and the formulations delineated herein, including glass, plastic, metal or polymer.

The invention further provides a method for treating, preventing or ameliorating TNFα or IL-1β related disorders by administering an effective amount of the Sophora flavescens flavonoids extract, its active ingredients (alone or in combination), and the compositions of the invention. A subject to be treated may be identified as being in need of inhibition of TNFα or IL-1β production. Identifying a subject in need of such treatment can be in the judgment of a subject or a health care professional, and can be subjective (e.g., opinion) or objective (e.g., measurable by a test or diagnostic method). The term “treating” is defined as administration of a substance to a subject with the purpose to cure, alleviate, relieve, remedy, prevent, or ameliorate a disorder, symptoms of the disorder, a disease state secondary to the disorder, or predisposition toward the disorder. An “effective amount” is an amount of the substance that is capable of producing a medically desirable result as delineated herein in a treated subject. The medically desirable result may be objective (i.e., measurable by some test or marker) or subjective (i.e., subject gives an indication of or feels an effect).

Examples of the TNFα related disorders to be treated, prevented or ameliorated include, but are not limited to, rheumatoid arthritis, juvenile rheumatoid arthritis, osteoarthritis, spondyloarthropathies, inflammatory bowel disease, chronic heart failure, diabetes mellitus, systemic lupus erythematosus, scleroderma, sarcoidosis, polymyositis/dermatomyositis, psoriasis, multiple myeloma, myelodysplastic syndrome, acute myelogenous leukemia, Parkinson's disease, AIDS dementia complex, Alzheimer's disease, depression, sepsis, pyoderma gangrenosum, hematosepsis, septic shock, Behcet's syndrome, graft-versus-host disease, uveitis, Wegener's granulomatosis, Sjogren's syndrome, chronic obstructive pulmonary disease, asthma, acute pancreatitis, periodontal disease, cachexia, cancer, central nervous system injury, viral respiratory disease, and obesity.

Examples of the IL-1β related disorders to be treated, prevented or ameliorated include, but are not limited to, rheumatoid arthritis, hematosepsis, periodontal disease, chronic heart failure, polymyositis/dermatomyositis, acute pancreatitis, chronic obstructive pulmonary disease, Alzheimer's disease, osteoarthritis, bacterial infections, multiple myeloma, myelodysplastic syndrome, uveitis, central nervous system injury, viral respiratory disease, asthma, depression, and scleroderma.

The effective amount of the Sophora flavescens flavonoids extract or its ingredients is between 1 and 300 mg/kg body weight per day. The effective amount can be any specific amount within the aforementioned range, wherein the lower boundary is any number of mg/kg body weight between 1 and 299, inclusive, and the upper boundary is any number of mg/kg body weight between 2 and 300, inclusive. The effective amount is useful in a monotherapy or in combination therapy for the treatment of TNFα and IL-1β related disorders. As the skilled artisan will appreciate, lower or higher doses than those recited above may be required. Effective amounts and treatment regimens for any particular subject (e.g., a mammal such as human) will depend upon a variety of factors, including the activity of the specific extract or its ingredients employed, the age, body weight, general health status, sex, diet, time of administration, rate of excretion, drug combination, the severity and course of the disease, condition or symptoms, the subject's disposition to the disease, condition or symptoms, and the judgment of the treating physician or veterinarian.

In order that the invention described herein may be more readily understood, the following examples are set forth. It should be understood that these examples are for illustrative purposes only and are not to be construed as limiting this invention in any manner. All references cited herein are expressly incorporated by reference in their entirety.

### EXAMPLE 1

### Preparation of a Sophora flavescens Flavonoids Extract

500 g dried plant material of Sophora flavescens Ait was soaked in 9 L 95% ethanol for three times. The ethanol extracts were combined and evaporated in vacuo to dryness. The 195 g residues were suspended in 30% ethanol aqueous solution and partitioned with ethyl acetate (1:1). The combined ethyl acetate extract was concentrated to provide 88.7 g total flavanoids (yield 45.5%).

### EXAMPLE 2

### Isolation and Identification of Three Ingredients in Sophora flavescens Flavonoids Extract

1. Isolation and Identification of Kurarinone

500 g dried plant material of Sophora flavescens was soaked in 3 L ethanol for three times. The ethanol extracts were combined and evaporated in vacuo to dryness. The 65 g residue was suspended in 6% methanol aqueous solution and partitioned with chloroform. The chloroform extract was concentrated and then subjected successively to LH-20 (eluent, CH₃OH), silica gel column (eluent, acetone/petroleum=1:2) and RP-18 column (eluent, methanol/water=65:35) to provide 2.0 g of Kurarinone (yield 0.4%).

The following spectral data was obtained for the isolated Kurarinon:

¹H NMR (DMSO-d6, 400 MHz): 10.2(2′-OH), 9.4(4′-OH), 9.2(7-OH), 7.4(1H, d, J=8.4 Hz, H-6′), 6.32(1H, d, J=2.28 Hz, H-3′), 6.25(1H, dd, J=8.2, 2.4 Hz, H-5′), 6.12(1H, s, H-6), 5.4(1H, dd, J=2.6, 13.1 Hz, H-2), 4.9(1H, t, J=1.4 Hz, H-4″), 4.55(1H, brs, H-9″a), 4.47(1H, brs, H-9″b), 3.7(3H, s), 2.8(1H, dd, J=13.1, 16.3 Hz, H-3a), 2.4(3H, m), 1.9(2H, m), 1.6(3H, s), 1.57(3H, s), 1.42(3H, s).

¹³C NMR (DMSO-d6, 75 MHz): 188.5, 162.1, 161.7, 159.2, 157.7, 154.9, 147.6, 130.3, 127.0, 123.1, 116.1, 110.5, 106.7, 106.0, 104.1, 102.1, 92.3, 73.4, 55.2, 46.3, 44.3, 30.7, 26.9, 25.5, 18.6, 17.6.

ESIMS (m/z): 439

2. Isolation and Identification of 2′-Methoxy-Kurarinone

500 g dried plant material of Sophora flavescens was soaked in 3 L ethanol for three times. The ethanol extracts were combined and evaporated in vacuo to dryness. The 65 g residue was suspended in 40% propanol aqueous solution and partitioned with ethyl ether. The ethyl ether extract was concentrated and then subjected successively to LH-20 (eluent, CH₃OH), silica gel column (eluent, acetone/petroleum=1:3) and RP-18 column (eluent, methanol/water=75:25) to provide 20 mg of 2′-methoxy-kurarinone (yield 0.01%).

The following spectral data was obtained for the isolated 2′-Methoxy-Kurarinone:

¹H NMR (CDCl3, 400 MHz): 7.4(1H, d, J=8.2 Hz, H-6′), 6.48(1H, dd, J=8.21, 2.34 Hz, H-5′), 6.44(1H, d, J=2.35 Hz, H-3′), 6.08(1H, s, H-6), 5.62(1H, m, H-2), 5.0(1 H, m), 4.72(1H, brs), 4.66(1H, brs), 3.8(3H, s), 3.76(3H, s), 2.84(1H, m, H-3a), 2.4(3H, m), 1.67(3H, s), 1.63(3H, s), 1.6(2H, m), 1.52(3H, s).

ESIMS (m/z): 451(M−1)

UV (MeOH) λₘₐₓ (logε) 286(4.3) nm,

IR (KBr) νₘₐₓ 3291, 2955, 2920, 1650, 1590, 1500, 1465, 1410, 1280 cm⁻¹.

3. Isolation and Identification of Sophoraflavanone G

500 g dried plant material of Kushen was soaked in 3 L ethanol for three times. The ethanol extracts were combined and evaporated in vacuo to dryness. The 65 g residue was suspended in 25% iso-propanol aqueous solution and partitioned with ethyl acetate. The ethyl acetate extract was concentrated and then subjected successively to LH-20 (eluent, CH₃OH), silica gel column (eluent, acetone/petroleum=1:3) and RP-18 column (eluent, methanol/water=70:30) to provide 300 mg Sophoraflavanone G (yield 0.06%).

The following spectral data was obtained for the isolated Sophoraflavanone G:

¹H NMR (DMSO-d6, 400 MHz): 12.1(s, 5-OH), 9.6(4′-OH), 9.4(7-OH), 7.22(1H, d, J=8.4 Hz, H-6′), 6.33(1H, d, J=2.3 Hz, H-3′), 6.26(1H, dd, J=8.4, 2.5 Hz, H-5′), 5.92(1H, s, H-6), 5.50(1H, dd, J=2.8, 13.3 Hz, H-2), 4.89(1H, t, J=6.8 Hz, H-4″), 4.55(1H, brs), 4.47(1H, brs), 3.1(1H, dd, J=13.3, 17.2 Hz, H-3a), 2.62(1H, dd, J=2.9, 17.2Hz, H-3b), 2.4(3H, m), 1.9(2H, m), 1.56(3H, s), 1.52(3H, s), 1.43(3H, s).

ESIMS (m/z): 423.

### EXAMPLE 3

### HPLC Analysis of Sophora flavescens Flavonoids Extract

The Sophora flavescens flavonoids extract prepared according to Example 1 was subjected to HPLC analysis using Agilent 1100 HPLC system with DAD detector under the following conditions: wavelength—280 nm, column—Zorbax SC18 4.6*150 mm, flow rate—1 ml/min, mobile phases—CH₃CN and H₂O, CH₃CN gradient—0 to 80% within 25 min, 80% to 100% within 5 min. The reference compounds of Kurarinone, 2′-Methoxy-Kurarinone and Sophoraflavanone G were each estimated to be more than 96% pure by HPLC analysis. The Sophora flavescens flavonoids extract was found to contain 29.1% Kurarinone (retention time=16.87 min), 1.8% 2′-methoxy-kurarinone (retention time=19.99 min), and 5.1% Sophoraflavanone G (retention time=20.65 min). See FIG. 3.

### EXAMPLE 4

### Inhibition Effects of Sophora flavescens Flavonoids Extract and Kurarinone on LPS-Induced Proinflammatory Cytokines in Normal Human PBMC

Materials:
1) Cell: PBMC (Peripheral Blood Monocytes)
2) Drugs: Sophora flavescens flavonoids extract and Kurarinone (Exp. 1)
3) Positive control: Dexamethason (DEX)
4) Reagents: Ficoll-Paque Plus (Amersham Bioscience), LPS and dexamethason (CalBiochem.), TNFa ELISA (Enzyme Linked Immunosorbent Assay) Kit and IL1-β ELISA Kit (Jingmei Bioengineer Technology), DMSO (Dimethylsulfoxide) (Sigma)

Method:

a. PBMC cells are isolated from fresh blood by using of Ficoll-Paque Plus reagent according to the protocol that the manufacturer recommends. Cells are suspended in RPMI 1640 media containing 10% FBS. 100 μl of 1×10⁵ cells/ml is seeded in each well of 96 well plate, 1×10⁴ cells total in each well and three wells for each reaction.

b. Samples are added into cells (final concentrations are 10, 30, 100 ug/ml, respectively. Sample volume that is added into each well is 10 μl). Final concentration of the positive control, dexamethason is 10 μM. 10 μl of media is added into the cells as a negative control. Then the plate is incubated in 37° C., 5% CO₂ incubator for 15 minutes, 10 μl of 100 μg/ml LPS was added to each well except for negative controls. Incubate in 37° C., 5% CO₂ incubator overnight.

c. Spin the plate at 1000 rpm for 15 minutes, transfer supernatant from each well to a new plate, measure the concentrations of TNFα and IL1-β by using assay kits.

$$Inhibition Ratio ( \% ) = ( 1 - \frac{Drug treatment - Control}{LPS treatment - Control} ) \times 100$$

Results:

TABLE 1
Inhibition effects of Sophora flavescens flavonoids extract
and Kurarinone on LPS-induced TNFα and IL1β.

| Dose | | TNFa | TNFa Inhibition | IL-1β | IL-1βInhibition |
|---|---|---|---|---|---|
| (μg/ml) | | (pg/ml) | Ratio (%) | (pg/ml) | Ratio (%) |
| Sophora flavescens | 10 | 129.1 ± 10.6 | 34.4 ± 10.1 | 184.9 ± 12.4 | 47.7 ± 10.1 |
| flavonoids extract | 30 | 50.9 ± 9.8 | 72.9 ± 2.1 | 76.9 ± 10.2 | 76.8 ± 2.1 |
| | 100 | 10.4 ± 0.4 | 94.3 ± 2.4 | 19.6 ± 0.4 | 93.9 ± 2.4 |
| Kurarinone | 10 | 92.2 ± 2.7 | 52.5 ± 4.2 | 129.8 ± 8.4 | 62.4 ± 4.2 |
| | 30 | 39.5 ± 1.5 | 78.5 ± 4.1 | 80.5 ± 3.7 | 75.0 ± 4.1 |
| | 100 | 2.1 ± 0.3 | 98.8 ± 2.1 | 3.1 ± 0.3 | 99.0 ± 0.5 |

| | | | | |
|---|---|---|---|---|
| PBMC | 11.0 ± 1.1 | / | 5.8 ± 1.0 | / |
| LPS | 215.7 ± 36.4 | / | 460.8 ± 16.4 | / |
| 10 uM DEX | 120.9 ± 3.4 | 61.3 ± 1.9 | 275.6 ± 5.3 | 72.9 ± 1.6 |

A number of embodiments of the invention have been described. Nevertheless, it will be understood that various modifications may be made without departing from the spirit and scope of the invention. Accordingly, other embodiments are within the scope of the following claims.

1. An extract of Sophora flavescens flavonoids, comprising 20˜60% by weight Kurarinone, 1˜5% by weight 2′-methoxy-Kurarinone, and 1˜12% by weight Sophoraflavanone G.

2. The extract of claim 1, consisting essentially of 20˜60% by weight Kurarinone, 1˜5% by weight 2′-methoxy-Kurarinone, and 1˜12% by weight Sophoraflavanone G.

3. The extract of claim 1, comprising 35˜45% by weight Kurarinone, 2˜4% by weight 2′-methoxy-Kurarinone, and 5˜8% by weight Sophoraflavanone G.

4. The extract of claim 3, consisting essentially of 35˜45% by weight Kurarinone, 2˜4% by weight 2′-methoxy-Kurarinone, and 5˜8% by weight Sophoraflavanone G.

5. The extract of claim 1, comprising 45% by weight Kurarinone, 2% by weight 2′-methoxy-Kurarinone, and 6% by weight Sophoraflavanone G.

6. The extract of claim 5, consisting essentially of 45% by weight Kurarinone, 2% by weight 2′-methoxy-Kurarinone, and 6% by weight Sophoraflavanone G.

7. A composition comprising isolated Kurarinone and a pharmaceutically acceptable carrier.

8. A composition comprising isolated 2′-methoxy-Kurarinone and a pharmaceutically acceptable carrier.

9. A composition comprising isolated Sophoraflavanone G and a pharmaceutically acceptable carrier.

10. A composition consisting essentially of at least two members of Kurarinone, 2′-methoxy-Kurarinone, or Sophoraflavanone G.

11. A composition comprising at least two members of Kurarinone, 2′-methoxy-Kurarinone, or Sophoraflavanone G, wherein at least a portion of one of the members in the composition is isolated.

12. The composition of claim 11, consisting essentially of at least two members of Kurarinone, 2′-methoxy-Kurarinone, or Sophoraflavanone G, wherein at least a portion of one of the members in the composition is isolated.

13. A method of inhibiting the expression of TNF-α in a subject in need thereof, comprising administering to said subject an effective amount of the extract of claim 1.

14. A method of inhibiting the expression of IL-1β in a subject in need thereof, comprising administering to said subject an effective amount of the extract of claim 1.

15. A method of inhibiting the expression of TNF-α in a subject in need thereof, comprising administering to said subject an effective amount of Kurarinone, 2′-methoxy-Kurarinone, Sophoraflavanone G, or a combination thereof.

16. A method of inhibiting the expression of IL-1β in a subject in need thereof, comprising administering to said subject an effective amount of Kurarinone, 2′-methoxy-Kurarinone, Sophoraflavanone G, or a combination thereof.

17. A method of inhibiting the expression of TNF-α in a subject in need thereof, comprising administering to said subject an effective amount of the composition of claim 11.

18. A method of inhibiting the expression of IL-1β in a subject in need thereof, comprising administering to said subject an effective amount of the composition of claim 11.

19. A method for treating TNFα related disease in a subject, comprising administering to a subject in need of such treatment an effective amount of the extract of claim 1.

20. A method for treating TNFα-related disease, comprising administering to a subject in need of such treatment an effective amount of Kurarinone, 2′-methoxy-Kurarinone, Sophoraflavanone G, or a combination thereof.

21. A method for treating TNFα related disease, comprising administering to a subject in need of such treatment an effective amount of the composition of claim 11.

22. The method according to claim 19, 20, or 21, wherein said TNFα-related disease is:
Juvenile rheumatoid arthritis;
Osteoarthritis;
Spondyloarthropathies;
Inflammatory bowel disease;
Chronic heart failure;
Diabetes mellitus;
Systemic lupus;
Erythematosus;
Scleroderma;
Sarcoidosis;
Polymyositis/dermatomyositis;
Psoriasis;
Multiple myeloma;
Myelodysplastic syndrome;
Acute myelogenous leukemia;
Parkinson's disease;
AIDS dementia complex;
Alzheimer's disease;
Depression;
Sepsis;
Pyoderma gangrenosum;
Hematosepsis;
Septic shock;
Behcet's syndrome;
Graft-versus-host disease;
Uveitis;
Wegener's granulomatosis;
Sjogren's syndrome;
Chronic obstructive pulmonary disease;
Asthma;
Acute pancreatitis;
Periodontal disease;
Cachexia;
Cancer;
Central nervous system injury;
Viral respiratory disease; or
Obesity.

23. A method for treating IL-1β related disease in a subject, comprising administering to a subject in need of such treatment an effective amount of the extract of claim 1.

24. A method for treating IL-1β related disease in a subject, comprising administering to a subject in need of such treatment an effective amount of Kurarinone, 2′-methoxy-Kurarinone, Sophoraflavanone G, or a combination thereof.

25. A method for treating IL-1β related disease in a subject, comprising administering to a subject in need of such treatment an effective amount of the composition of claim 11.

26. The method according to claim 23, 24, or 25, wherein said IL-1β related disease is:
Rheumatoid arthritis;
Hematosepsis;
Periodontal disease;
Chronic heart failure;
Polymyositis/dermatomyositis;
Acute pancreatitis;
Chronic obstructive pulmonary disease;
Alzheimer's disease;
Osteoarthritis;
Bacterial infections;
Multiple myeloma;
Myelodysplastic syndrome;
Uveitis;
Central nervous system injury;
Viral respiratory disease;
Asthma;
Depression; or
Scleroderma.

27. A method of producing an extract of Sophora flavescens flavonoids of claim 1, the method comprising:
soaking the plant material of Sophora flavescens Ait in organic solvent to give an extract;
evaporating the extract in vacuo to dryness to give an extract residue;
dissolving the extract residue in 5%˜50% C₁˜C₄ alcohol aqueous solution to give an extract alcohol solution;
extracting the extract alcohol solution with organic solvent to give organic extracts;
and concentrating the organic extracts.

28. The method according to claim 27, wherein said C₁˜C₄ alcohol aqueous solution is selected from the group consisting of methanol, ethanol, propanol, iso-propanol or butanol.

29. The method according to claim 27, wherein the concentration of said C₁˜C₄ alcohol aqueous solution is 20˜40%.

30. The method according to claim 29, wherein the concentration of said C₁˜C₄ alcohol aqueous solution is about 30%.

31. A method of producing a composition of claim 7, the method comprising combining isolated Kurarinone with a pharmaceutically acceptable carrier.

32. A method of producing a composition of claim 8, the method comprising combining isolated 2′-methoxy-Kurarinone with a pharmaceutically acceptable carrier.

33. A method of producing a composition of claim 9, the method comprising combining isolated Sophoraflavanone G with a pharmaceutically acceptable carrier.

34. A method of producing a composition of claim 11, the method comprising combining at least two members of Kurarinone, 2′-methoxy-Kurarinone, or Sophoraflavanone G, wherein at least a portion of one of the members in the composition is isolated.

35. A method of inhibiting TNF-α production in an isolated cell that produces TNF-α comprising contacting an extract of claim 1 with the cell.

36. A method of inhibiting TNF-α production in an isolated cell that produces TNF-α comprising contacting a composition of any of claims 7-11 with the cell.

37. A method of inhibiting IL-1β production in an isolated cell that produces IL-1β comprising contacting an extract of claim 1 with the cell.

38. A method of inhibiting IL-162 production in an isolated cell that produces IL-1β comprising contacting a composition of any of claims 7-11 with the cell.
